(19) United States
(12) Patent Application Publication
(10) Pub. No.: US 2007/0060888 A1
(43) Pub. Date: Mar. 15, 2007

(54) Methods and apparatus for assisted aspiration

(75) Inventors: Thomas G. Goff, Menlo Park, CA (US); Michael J. Orth, Morgan Hill, CA (US); Mark C. Yang, San Francisco, CA (US); Hung Ha, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: KERBEROS PROXIMAL SOLUTIONS, INC., Cupertino, CA (US)

(21) Appl. No.: 11/220,874

(22) Filed: Sep. 6, 2005

(51) Int. Cl. A61M 1/00

(52) U.S. Cl. 604/118; 604/43; 604/528

(57) ABSTRACT

An aspiration catheter comprises a catheter body having an aspiration lumen therethrough. The distal opening of the aspiration lumen can be modified in various ways or can have the addition of structure in order to disrupt sealing between the port and material being aspirated. Examples of modifications include slots, castellations, zig-zag features, and other geometries which resist sealing of aspirated material. Blades, rollers, vibrating elements, and other structures may be provided within or around the distal end of the aspiration lumen in order to dislodge and disrupt aspirated material which can seal against the opening.

### BACKGROUND OF THE INVENTION

1. Field of the Invention.

The present invention relates generally to medical apparatus and methods. More particularly, the present invention relates to the construction and use of aspiration catheters which are modified to disrupt the formation of a seal between a distal aspiration port and the material being aspirated.

Coronary and peripheral vascular disease are often characterized by the partial or complete blockage of a blood vessel by plaque, thrombus, emboli, or other substances which occupy the lumen of the blood vessel. Such partial or complete blockage can cause ischemia which can lead to heart attacks, gangrene in the limbs, and other life threatening conditions.

A number of different catheter-based technologies have been developed to treat such vascular obstructions. Of particular interest to the present invention, the use of aspiration catheters for removing relatively soft thrombus and clot has been proposed. An aspiration catheter typically comprises a tubular body having at least one aspiration lumen therein. By applying a vacuum at a proximal end of the lumen, clot, thrombus and other occlusive materials can be drawn into a distal port and removed from the blood vessel or other body lumen.

A significant shortcoming of the use of such simple aspiration catheters is the tendency for such catheters to plug or seal against the material which is being removed. Should the distal port on the catheter become plugged or sealed, it will often be necessary to remove the catheter, clear the plugging, and then return the catheter to the site of intervention. The need to remove and clear the catheter can arise more than once in any procedure, significantly slowing down the procedure and increasing risk of trauma to the patient.

For these reasons, it would be desirable to provide improved aspiration catheters and methods for their use, where the aspiration catheter would have a reduced likelihood of clogging or sealing when removing clot, thrombus, or other substances from blood vessels and other body lumens. It would be particularly desirable to provide catheter designs which are inherently unlikely to occlude during use. Alternatively or additionally, it would be beneficial to provide methods and protocols for performing aspiration with such catheters, where the methods are less likely to result in occlusion and/or allow for the convenient disruption of any seal or occlusion which may form during use. Additionally, it would be desirable to provide catheter design features and/or methods for using such catheters where the features or methods could be combined to provide multiple approaches for lessening the risk of catheter plugging and/or facilitating unplugging of catheters when they have become plugged. At least some of these objectives will be met by the inventions described herein below.

2. Description of the Background Art.

Catheters and systems for aspirating clot and other materials for blood vessels and other body lumens are described in U.S. Pat. Nos. 6,849,068; 6,468,262; 5,938,645; 5,827,243; 5,749,858; 5,569,204; and 3,937,220.

### BRIEF SUMMARY OF THE INVENTION

The present invention provides for improved methods and apparatus for luminal aspiration to remove substances which partially or fully occlude the lumen being treated. Although particularly useful for aspirating clot, thrombus, and other soft materials from arteries and veins, the methods and apparatus of the present invention will also find use for removing potentially occlusive and other materials from other body lumens, such as the ureter, urethra, bowel, colon, ear canal, nasal passages, sinuses, and the like.

Aspiration catheters according to the present invention comprise a catheter body and a hub. The catheter body has a distal end, a proximal end, and an aspiration lumen therethrough, and the aspiration lumen terminates in a distal aspiration port. A hub is disposed at the proximal end of the catheter body and is adapted to connect the aspiration lumen to a vacuum source. The distal aspiration port will be modified in some way to disrupt potential sealing between the port and the material being aspirated. Often, the catheter body will have a separate guidewire lumen running at least part of the way there though in order to facilitate introduction to blood vessels in an “over-the-wire” manner. Optionally, the catheter body may further include an infusion lumen, and the infusion lumen may terminate in one or more side infusion port(s) near the distal end of the catheter body. The provision of both an aspiration port and one or more infusion ports allows simultaneous infusion and aspiration which can be beneficial for many treatment protocols.

The aspiration port may be modified in a variety of ways in order to disrupt potential sealing between the port and the material being aspirated. In a first example, a disruption element may be provided near or within the aspiration port in order to break up material as it is drawn into the aspiration port by an applied vacuum. The disruption element may be passive, for example being a fixed wire or blade located near or across the aspiration lumen. The use of such a wire or blade will physically break up the clot as or while it is being drawn into the aspiration port, thus reducing the risk that the material will partially or fully occlude and block the aspiration port as it is being drawn in.

Alternatively, the disruption element could be an active element, i.e. one where the element is powered and/or free to move or change positions relative to the aspiration port. For example, the active of element may comprise at least one vibration wire which may be driven, for example, by a piezoelectric element or other vibrating member. The active element could also comprise one or more roller(s) mounted along or over the periphery of the port. Such rollers will physically disrupt sealing of the material being drawn into the port while allowing the material to pass freely over the roller, i.e. the roller “rolls” and reduces friction as the material as being drawn into the port.

Alternatively, the disruption element could be a semi-active element which is driven or powered by a flow of blood or other fluid. For example, the wire element 66 shown in FIG. 10 discussed below could be constructed so that vibration or other movement is induced by a passing flow of fluid. Such flow could be the result of aspiration of the system. As blood and debris are aspirated, the flow of the aspirate past the element could impart energy into the element causing a vibration or other movement that would further disrupt the debris.

The port modification may also comprise modifications of the geometries, shapes, profiles, or other geometrical characteristics of the port. For example, the periphery or lip of the port may have one or more axial slots formed therein. Such slots allow the bypass of infused materials should a larger clot, thrombus, or other material become lodged in the main region of the port. Alternatively, the lip or periphery of the port may have an irregular edge, such as a castellated edge, a zig-zag edge, a serpentine edge, or other shapes which reduce the likelihood that the material will seal completely and continuously around the lip of the port.

In a related concept, the port modification could comprise one or more side apertures adjacent to and optionally surrounding the aspiration port. The side apertures may be open or have one-way or other valves in them which permit the bypass aspiration of blood or other body fluids into the aspiration catheter when the main area of the aspiration port has become plugged. The one-way valves could open in response to aspiration of the aspiration lumen, in response to pressurization of the aspiration lumen, or there maybe some valves which respond in each of the aforesaid manners.

In addition to structural modifications in the aspiration port, the aspiration catheter can be used in a system and with a protocol which permits delivery of a “pressure pulse” to disrupt a distal seal which is formed around the periphery of the aspiration port. Such “pulse pressurization” will typically result from varying the negative pressure of the vacuum being applied. That is, the pressure pulse may simply be one or more reductions and/or increase in the level of vacuum being applied through the port. In other instances, however, the pressure pulse could be above ambient so that any material lodged within the aspiration port would be expelled by the higher pressure. In particular instances, the catheters of the present invention may be provided with separate “pulse” lumens in order to allow for pressure pulses to be delivered other than through the aspiration lumen.

In yet another embodiment, a fluid lumen, typically the infusion lumen but alternatively a separate dedicated lumen, directs fluid flow to the aspiration port in such a manner to prevent the clogging or occlusion of the tip and/or to dislodge or unclog material from the tip. This fluid jet(s) could be delivered on the interior or exterior of the aspiration lumen and should provide a force sufficient to disrupt a seal and/or break up a thrombus or other biological material being aspirated. The infusion fluid could be supplemented with agents (e.g., thrombolytics or other drugs) to further enhance the efficacy of the system.

The present invention still further provides methods for aspirating clot and other occlusive material from blood vessels and other body lumens. The methods comprise engaging the clot or other material with an aspiration port of an aspiration catheter. A vacuum is applied to an aspiration lumen in the catheter to draw the material into the lumen through the aspiration port. Any seals or plugging between the material and the port would be disrupted in order to permit the aspiration to continue. Such disruption may comprise breaking the material as it is drawn into the aspiration lumen, e.g. using the fixed or active disruption elements described above. The disruption could alternatively comprise active disruption, e.g. vibrating a wire or rotating a rotatable element adjacent to the aspiration port. The disruption could still further comprise providing gaps or apertures at or near the lip of the aspiration port. Still further, disrupting the sealing may comprise providing a pressure pulse as clot enters the aspiration port. The pressure pulse may comprise a variation and the negative pressure of the vacuum being applied or alternatively may comprise applying a positive pressure, either through the aspiration lumen or through a separate pressurization lumen provided in the aspiration catheter.

### BRIEF DESCRIPTION OF THE DRAWINGS

FIG. 1 illustrates an exemplary aspiration catheter according to the present invention including an aspiration lumen, a pressure pulse lumen, and a guidewire lumen.

FIGS. 2A-2C illustrate use of the aspiration catheter of FIG. 1 for aspirating thrombus and disrupting a seal between the distillation port and the thrombus in accordance with the principles of the present invention.

FIG. 3 illustrates modification of the aspiration port of the catheter of FIG. 1 to include axial slots.

FIG. 4 illustrates modification of the aspiration port the catheter of FIG. 1 to include sealing disruption ports.

FIG. 5 illustrates modification of the aspiration port of the aspiration port of the catheter of FIG. 1 to include castellations.

FIG. 6 illustrates modification of the aspiration port of the catheter of FIG. 1 to include a zig-zag periphery.

FIG. 7 illustrates a simplified aspiration catheter having sealing-disruption fingers distributed about periphery of the aspiration port.

FIG. 8 illustrates a clot disruption blade disposed across the aspiration port of a catheter according to the present invention.

FIGS. 9A and 9B illustrate the provision of rolling elements about the periphery of a distal aspiration port according to the present invention.

FIG. 10 illustrates inclusion of a vibrating element in the distal aspiration port of a catheter according to the present invention.

FIGS. 11A and 11B. illustrate an alternative embodiment of a roller element for disrupting clot as it enters the port of an aspiration catheter in accordance with the principles of the present invention.

FIG. 12 illustrates a side flap for disrupting a seal between clot in an aspiration port in a catheter according to the present invention.

FIG. 13 illustrates a forwardly disposed blade over the distal aspiration port of a catheter in accordance with the principles of the present invention.

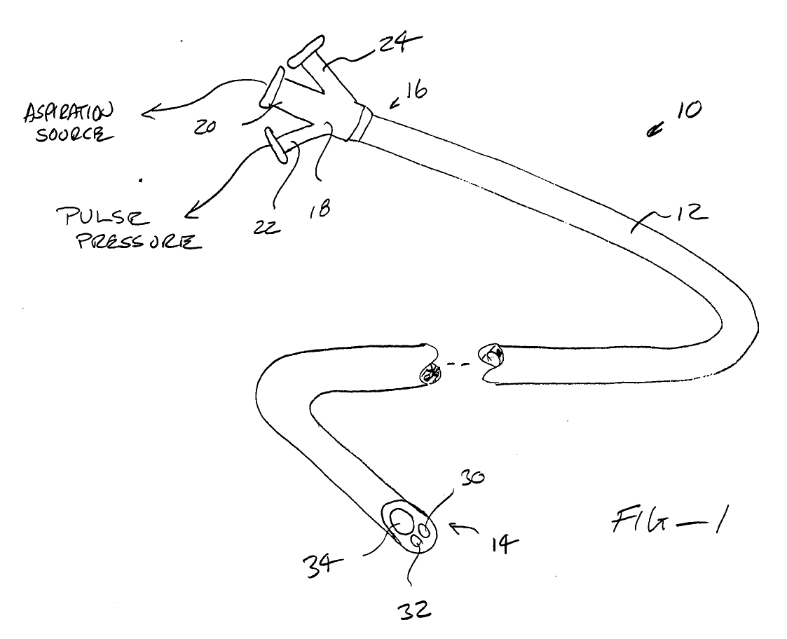

### DETAILED DESCRIPTION OF THE INVENTION

An aspiration catheter 10 constructed in accordance with the principles of the present invention comprises a catheter body 12 having a distal end 14 and proximal end 16. A proximal hub 18 is attached to the proximal end 16 of the catheter body 12 and includes an aspiration connector 20, a pulse lumen connector 22, and a guidewire connector 24. The aspiration connector 20 will be adapted to connect to a vacuum source, typically operating at from minus 10 mmHg to minus 760 mmHg. A pulse lumen connector, which is optional, may be attached to a pressure source, typically operating from positive 10 mmHg to positive 760 mmHg.

The guidewire connection 24 allows the guidewire to enter guidewire lumen 30 and the catheter body 12, while the pulse pressure connector 22 connects to the pulse pressure lumen 32. The aspiration connector 20 connects to the central aspiration lumen 34. In this way, while the aspiration lumen 34 is connected to the aspiration source, the guidewire can be introduced over a guidewire and optionally, pressure pulses may be delivered through the pressure pulse lumen 32 in order to break a seal and dislodge clot which may have become plugged in the distal end of the aspiration lumen 34.

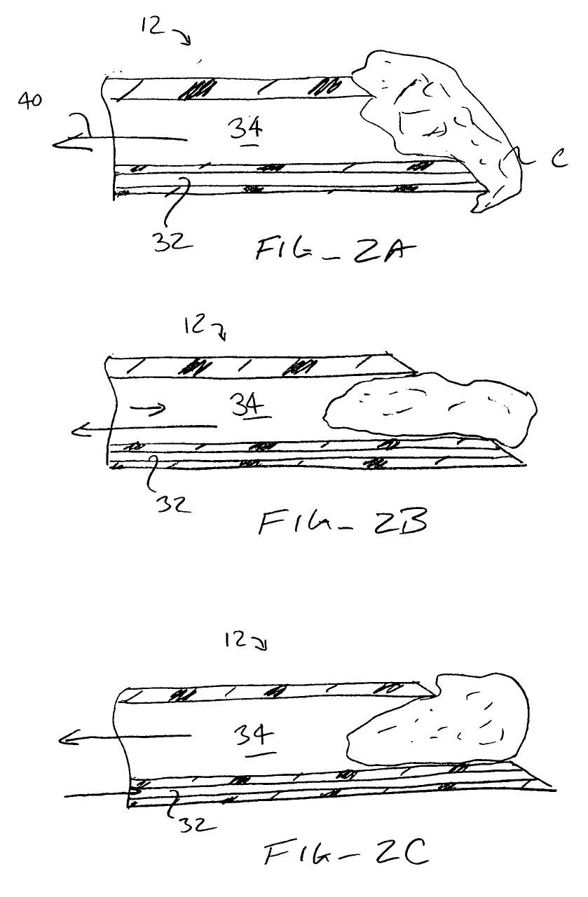

Referring now to FIGS. 2A-2C, clot C is generally drawn into the aspiration lumen 34 when a vacuum is applied to the lumen, as indicated by arrow 40. In some cases, the clot C may form a cap or plug over the distal end of the aspiration lumen 34, as shown in FIG. 2B. When that occurs, the aspiration vacuum may be manipulated in various ways to break the seal which is formed and to help dislodge the clot, allowing it to be drawn into the aspiration lumen 34 and removed. As shown in FIG. 2B, the aspiration pressure may be varied in order to dislodge the clot. Usually, the pressure being applied through the aspiration lumen 34 will remain negative, but the level of the vacuum being applied will be varied in an oscillatory or other pattern. Such oscillation or other variation in pressure helps break the seal and plug which has formed and draws the clot into the lumen, as shown in FIG. 2B.

Alternatively or additionally, an optional pressure pulse lumen 32 may be used to deliver a positive pressure pulse against the clot which has formed over the distal end of the catheter body 12, as shown in FIG. 2C. Such separate pressure pulses can also act to dislodge the clot and break the seal, allowing the clot to be drawn into the aspiration lumen 34.

While the use of pressure variations is one technique used by the present invention to dislodge and disrupt sealed clot, the present invention further provides for a number of active and passive structures and modifications at the distal opening of the aspiration port 34. For example, as shown in FIG. 3, a plurality of slots 50 may be formed around all or a portion of the distal end of the lumen 34. These slots will permit the aspiration of fluid even when the distal end of the lumen 34 has been plugged. The continuing flow of fluid into the lumen will often be able to dislodge the clot and allow it to be drawn fully into the aspiration lumen. Similarly, a plurality of side ports 52 may be formed in the wall of catheter body 12 allowing the inflow of aspirated fluid even when the distal end of the lumen 34 has been completely covered by clot.

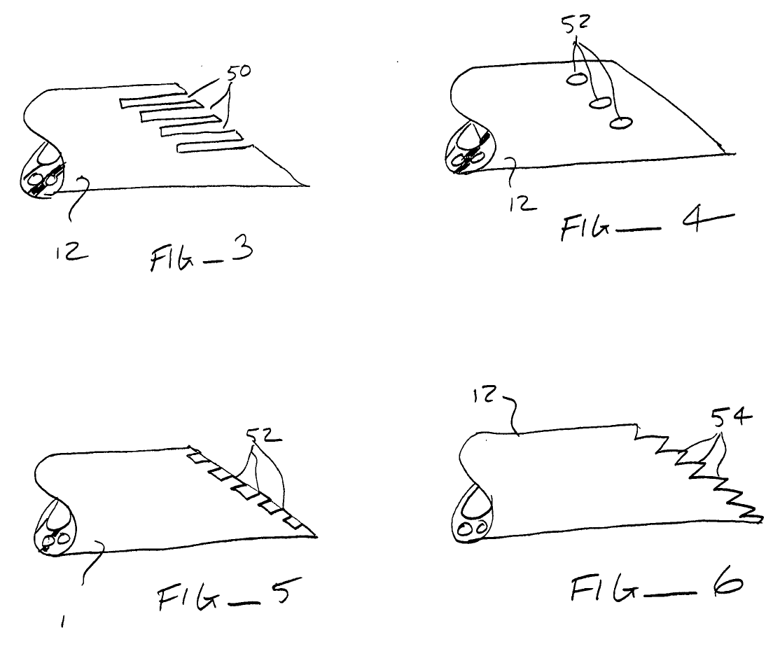

FIGS. 5 and 6 show alternative approaches where the periphery about the distal port of the aspiration lumen 34 is modified so that it is discontinuous. For example, castellations 54 may be formed about the distal end of the aspiration lumen, making it more difficult for a sealed form. Similarly, the periphery of the aspiration lumen may be modified to have a plurality of zig-zag protrusions 54 (as shown in FIG. 6) or may be otherwise modified and have a discontinuous edge which resists the formation of an occlusive seal of clot.

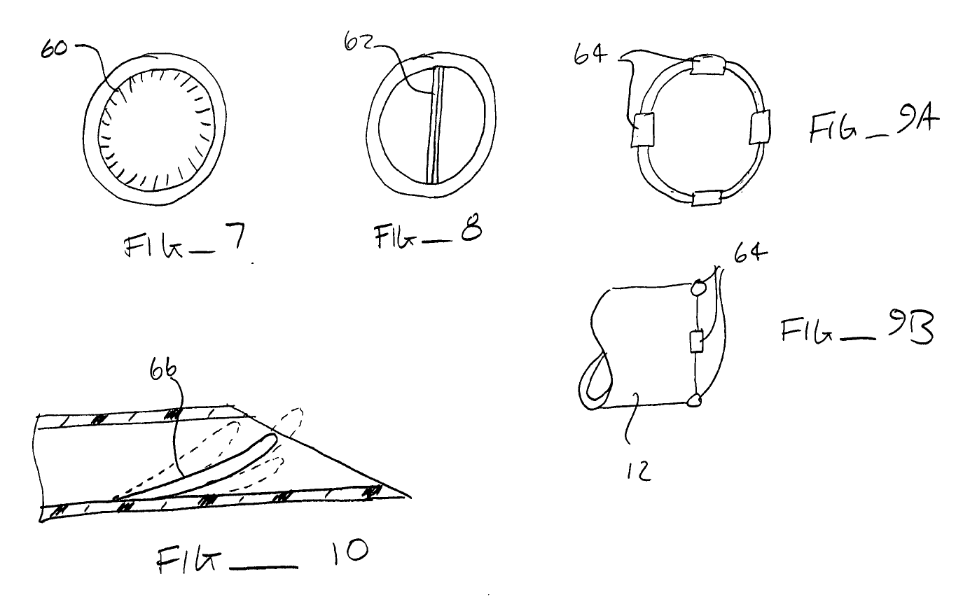

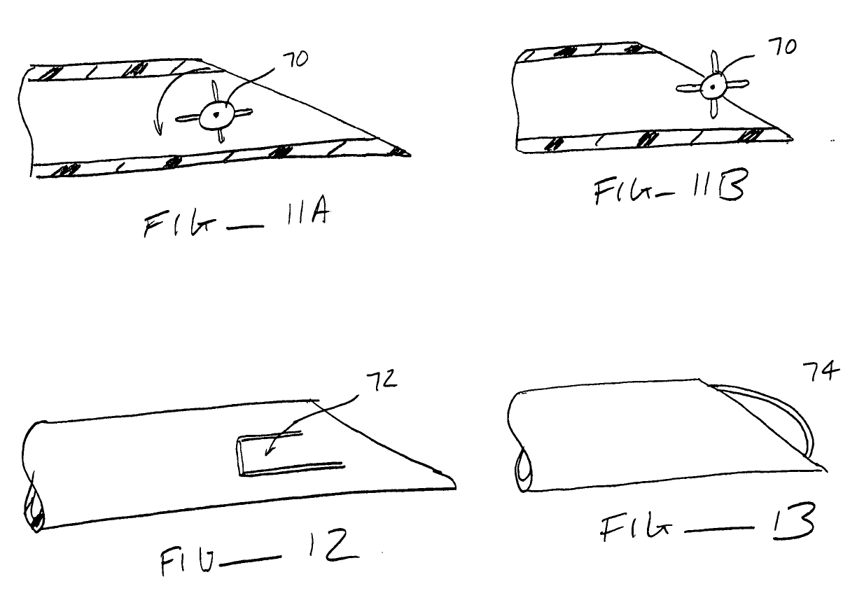

Other passive features which may be incorporated into the distal end or port of the aspiration lumen include plurality of fingers or cilia 60 which may be formed to intrude into the distal opening of the lumen (FIG. 7), a cutting blade or wire 62 which may extend across the opening of the lumen (FIG. 8), a plurality of rollers 64 which may be distributed about the periphery of the aspiration lumen (FIGS. 9A and 9B), and a vibrating finger 66 which may be disposed at or recessed within the distal opening of the aspiration lumen (FIG. 10). Further structures which may be provided include a roller structure having paddles 70 which may be recessed within the distal opening of the aspiration lumen (FIG. 11A) or may extend slightly outside of the distal end (FIG. 11B). A side flap 72 may also be formed in the wall of the aspiration lumen and allow for breaking of the vacuum seal which may be formed (FIG. 12). Additionally, a wire or cutting element 74 may be bowed outwardly from the distal opening of the aspiration lumen in order to disrupt clot as it enters (FIG. 13).

While the above is a complete description of the preferred embodiments of the invention, various alternatives, modifications, and equivalents may be used. Therefore, the above description should not be taken as limiting the scope of the invention which is defined by the appended claims.

What is claimed is:

1. An aspiration catheter comprising:
a catheter body having a distal end, a proximal end, and an aspiration lumen therethrough, wherein the aspiration lumen terminates at a distal aspiration port;
a hub at the proximal end of the catheter body, said hub adapted to connect the aspiration lumen to a vacuum source; and
means at the distal aspiration port for disrupting sealing between the port and material being aspirated.

2. An aspiration catheter as in claim 1, wherein the catheter body also has a guide wire lumen.

3. An aspiration catheter as in claim 2, wherein the catheter body also has an infusion lumen.

4. An aspiration catheter as in claim 3, wherein the infusion lumen terminates in side infusion ports near the distal end of the catheter body.

5. An aspiration catheter as in any one of claims 1 to 4, wherein the sealing disruption means comprises a disruption element within the aspiration port.

6. An aspiration catheter as in claim 5, wherein the element is passive.

7. An aspiration catheter as in claim 5, wherein the passive element comprises at least one wire or blade.

8. An aspiration catheter as in any one of claims 1 to 4, wherein the sealing disruption means comprises an active or driven element.

9. An aspiration catheter as in claim 8, wherein the active element comprises at least one vibration wire.

10. An aspiration catheter as in claim 8, wherein the active element comprises at least one roller mounted along a periphery of the port.

11. An aspiration catheter as in any one of claims 1 to 4, wherein the sealing disruption means comprises an irregular aspiration port lip.

12. An aspiration catheter as in claim 11, wherein the lip has at least one axial slot.

13. An aspiration catheter as in claim 11, wherein the lip has an irregular edge.

14. An aspiration catheter as in claim 13, wherein the irregular edge is castellated.

15. An aspiration catheter as in claim 13, wherein the irregular edge is zig-zag.

16. An aspiration catheter as in any one of claims 1 to 4, wherein the sealing disruption means comprises one or more side apertures adjacent to the aspiration port.

17. An aspiration catheter as in claim 16, wherein the side apertures are open.

18. An aspiration catheter as in claim 16, wherein at least some of the side apertures have cover valves.

19. An aspiration catheter as in claim 18, wherein the cover valves open in response to aspiration of the aspiration lumen.

20. An aspiration catheter as in claim 18, wherein the cover valves open in response to pressurization of the aspiration lumen.

21. An aspiration catheter as in any one of claims 1 to 4, wherein the sealing disruption means comprises a pulse lumen to deliver pressurized fluid to disrupt a distal seal.

22. An aspiration catheter as in claim 21, wherein the pulse lumen is a separate tube.

23. An aspiration catheter as in claim 21, wherein the pulse lumen is formed in a wall of the catheter body.

24. A method for aspirating clot from a blood vessel, said method comprising:
engaging clot with an aspiration port of an aspiration catheter,
applying a vacuum to an aspiration lumen in the catheter to draw clot into the lumen and through the aspiration port, and
disrupting sealing between the clot and the port as the clot is drawn into the lumen.

25. A method as in claim 24, wherein disrupting comprises breaking up clot as it is drawn into the aspiration lumen.

26. A method as in claim 25, wherein breaking up the clot results from a fixed wire or blade placed at the aspiration port.

27. A method as in claim 25, wherein disrupting clot comprises moving a disruption element at the aspiration port.

28. A method as in claim 27, wherein moving comprises vibrating a wire or rotating a roller adjacent to the aspiration port.

29. A method as in claim 24, wherein disrupting sealing comprises providing gaps or apertures in a lip of the distal aspiration port.

30. A method as in claim 24, wherein disrupting sealing comprises pressure pulsing clot as it enters the aspiration port.

31. A method as in claim 30, wherein the pressure pulses comprise vacuum variation.

32. A method as in claim 30, wherein the pressure pulses comprise positive pressure variations.
